(19) United States
(12) Patent Application Publication
(10) Pub. No.: US 2025/0121022 A1
(43) Pub. Date: Apr. 17, 2025

(54) ALPINIAE OFFICINARUM RHIZOME EXTRACT, PREPARATION METHOD AND USE THEREOF

(71) Applicant: Infinitus (China) Company Ltd., Jiangmen (CN)

(72) Inventors: Yaqian GAO, Jiangmen (CN); Jian TANG, Jiangmen (CN); Na ZHAO, Jiangmen (CN); Yunlin ZENG, Jiangmen (CN)

(21) Appl. No.: 18/525,585

(22) Filed: Nov. 30, 2023

(30) Foreign Application Priority Data
Oct. 13, 2023 (CN) 202311329123.5

(51) Int. Cl. A61K 36/9062

(52) U.S. Cl. CPC A61K 36/9062; A61K 2236/15; A61K 2236/39; A61K 2236/51

(57) ABSTRACT

Disclosed are an Alpiniae Officinarum Rhizome extract, a preparation method and use thereof. The preparation method comprises: S1: adding vegetable oil into an acidic mixed solution of Alpiniae Officinarum Rhizome superfine powder and stirring a resulting mixture, and then removing the vegetable oil to obtain an Alpiniae Officinarum Rhizome precipitate; S2: adding an emulsifier and a filler into an acidic mixed solution of the Alpiniae Officinarum Rhizome precipitate, and homogenizing a resulting mixture to obtain a homogeneous solution of Alpiniae Officinarum Rhizome, and S3: grinding and then drying the homogeneous solution of Alpiniae Officinarum Rhizome to obtain the Alpiniae Officinarum Rhizome extract. The preparation technology of the present disclosure solves the problems of a large amount of solvent and harsh processing condition of a traditional technology, and simultaneously reduces the problem of strong acrid taste of the Alpiniae Officinarum Rhizome extract, thus being suitable for industrial popularization and application.

### CROSS-REFERENCE TO RELATED APPLICATION

The present disclosure is based on and claims the benefit of and priority to Chinese Patent Application No. 202311329123.5, filed on Oct. 13, 2023, the entire contents of which are incorporated herein by reference.

### TECHNICAL FIELD

The present disclosure relates to the technical field of natural extracts, and particularly to an Alpiniae Officinarum Rhizome extract, a preparation method and use thereof.

### BACKGROUND

Alpiniae officinarum Rhizome comes from “Supplementary Records of Famous Physicians”, and is a dried rhizome of Alpinia Officinarum Hance. Alpiniae officinarum Rhizome is hot in nature and acrid in taste, has the effects of warming stomach, dispelling cold, promoting digestion and relieving pain, and is often used for treating abdominal cold pain, stomach cold vomiting, belching and acid swallowing, and other symptoms. Modern researches show that Alpiniae Officinarum Rhizome mainly contains flavonoid, diphenyl heptane and volatile oil ingredients, and the acrid taste of the Alpiniae Officinarum Rhizome and an extract thereof mainly comes from a diphenyl heptane compound, which has the effects of anti-tumor, anti-oxidation, anti-virus, anti-bacterium, treatment of gastric ulcer and protection of gastric mucosa. The most commonly used part of the Alpiniae Officinarum Rhizome is the rhizome thereof, which is acrid in taste and aromatic in smell, and the rhizome of the Alpiniae Officinarum Rhizome has always been used as a spice or a condiment. An Alpiniae Officinarum Rhizome extract prepared by a traditional technology has a strong acrid taste, which greatly affects an edible taste, thus limiting its application in medicine, health care and functional food, and its dosage form selection. Therefore, it is necessary to optimize the preparation technology of the Alpiniae Officinarum Rhizome extract to reduce the acrid taste of the Alpiniae Officinarum Rhizome extract and ensure that a content of an active ingredient of galangin is not affected at the same time, which is of great significance to the application of the Alpiniae Officinarum Rhizome extract in the food field.

In the prior art, the galangin is mainly obtained by a solvent extraction method, and a large amount of solvent is used in a technological process, which has disadvantages of a high technological cost and environmental unfriendliness. In other prior arts, the preparation is carried out at a high temperature, and technological conditions are harsh, thus being not beneficial for industrial processing and production.

### SUMMARY

The present disclosure aims to solve at least one of the above-mentioned technical problems in the prior art. Therefore, the present disclosure aims to provide an Alpiniae Officinarum Rhizome extract, a preparation method and use thereof. The preparation method of the Alpiniae Officinarum Rhizome extract as described in the present disclosure can overcome the current defect of strong acrid taste of Alpiniae Officinarum Rhizome or an extract thereof, or the above defect of the processing technology thereof, and uses the raw and auxiliary materials allowed by the food law. Moreover, in the present disclosure, the preparation technology of the Alpiniae Officinarum Rhizome is processed and optimized by an in-situ emulsification technology, so as to improve a content of galangin in the product, and eliminate or reduce the acrid taste of the product to the greatest extent, thus obtaining the Alpiniae Officinarum Rhizome extract with a suitable taste.

In order to achieve the above objectives, the technical solution used in the present disclosure is as follows.

In a first aspect of the present disclosure, a preparation method of an Alpiniae Officinarum Rhizome extract is provided, which comprises the following steps of:
S1: adding vegetable oil into an acidic mixed solution of Alpiniae Officinarum Rhizome superfine powder and stirring a resulting mixture, and then removing the vegetable oil to obtain an Alpiniae Officinarum Rhizome precipitate;
S2: adding an emulsifier and a filler into an acidic mixed solution of the Alpiniae Officinarum Rhizome precipitate, and homogenizing a resulting mixture to obtain a homogeneous solution of Alpiniae Officinarum Rhizome; and
S3: grinding and then drying the homogeneous solution of Alpiniae Officinarum Rhizome to obtain the Alpiniae Officinarum Rhizome extract.

In the present disclosure, the vegetable oil has a limited permeability to the Alpiniae Officinarum Rhizome, so that the extraction needs to be carried out with the Alpiniae Officinarum Rhizome superfine powder, which can not only effectively reduce ingredients in the extract leading to the acrid taste, such as volatile oil, 1′-acetoxychavicol acetatee, diphenylheptane A and galangol in the Alpiniae Officinarum Rhizome; but also retain the galangin to the greatest extent, thus increasing the content of the galangin in the extract. The combination of Alpiniae Officinarum Rhizome superfine powder and an emulsification technology can prepare the Alpiniae Officinarum Rhizome superfine powder into a wrapped form, so as to improve the stability. Moreover, the galangin is an organic substance with two phenolic hydroxyl groups and one enol hydroxyl group, which is easily oxidized in a solution/amorphous system, and has extremely poor solubility in other organic solvents. By using other organic solvents such as petroleum ether, only the volatile oil ingredient of Alpiniae Officinarum Rhizome can can be removed, which has little effect on its acrid taste. However, the above main ingredients leading to the acrid taste of the Alpiniae Officinarum Rhizome have high solubility in the vegetable oil, the galangin has low solubility in the vegetable oil at the same time, thus a difference in solubility makes the extraction method of the present disclosure have selectivity, and the obtained Alpiniae Officinarum Rhizome extract has a low acrid taste and a high content of galangin.

In some embodiments of the present disclosure, in the step S1, a preparation method of the acidic mixed solution of the Alpiniae Officinarum Rhizome superfine powder comprises the following steps of: crushing the Alpiniae Officinarum Rhizome, sieving a crushed Alpiniae Officinarum Rhizome with an 80-mesh to 150-mesh sieve, and then carrying out superfine crushing on a sieved Alpiniae Officinarum Rhizome to prepare the Alpiniae Officinarum Rhizome superfine powder; and adding an acidic solution to the Alpiniae Officinarum Rhizome superfine powder according to a solid-liquid ratio of 1 g: (6 to 12) mL, and sealing and standing a resulting mixture to prepare the acidic mixed solution of the Alpiniae Officinarum Rhizome superfine powder. Preferably, a pH value of the acidic solution is 3.5 to 5.5; preferably, the pH value of the acidic solution is regulated to be 3.5 to 5.5 with an acidity regulator; preferably, the acidity regulator comprises at least one of citric acid, tartaric acid, malic acid and acetic acid, and preferably, the standing lasts for 30 minutes to 180 minutes.

In some embodiments of the present disclosure, in the step S1, a solid-liquid ratio of the Alpiniae Officinarum Rhizome superfine powder to the vegetable oil is 1:(0.5 to 3).

In some embodiments of the present disclosure, the vegetable oil comprises at least one of peanut oil, olive oil, safflower seed oil, soybean oil, corn oil and rapeseed oil.

In some embodiments of the present disclosure, in the step S1, after adding the vegetable oil into the acidic mixed solution of Alpiniae Officinarum Rhizome superfine powder and stirring the resulting mixture, sealing and standing the resulting mixture for 1 hour to 5 hours (such as 2 hours, 3 hours, and 4 hours, and the like), and then performing centrifugation to move vegetable oil to obtain the Alpiniae Officinarum Rhizome precipitate.

In some embodiments of the present disclosure, in the step S2, a solid-liquid ratio of the acidic mixed solution of the Alpiniae Officinarum Rhizome precipitate is 1 g: (6 to 12) mL. Preferably, a preparation method of the acidic mixed solution of the Alpiniae Officinarum Rhizome precipitate comprises the following step of: adding the Alpiniae Officinarum Rhizome precipitate into an acidic solution according to the solid-liquid ratio of 1 g: (6 to 12) mL; preferably, a pH value of the acidic solution is 3.5 to 5.5; preferably, the pH value of the acidic solution is regulated to be 3.5 to 5.5 with an acidity regulator; and preferably, the acidity regulator comprises at least one of citric acid, tartaric acid, malic acid and acetic acid.

In some embodiments of the present disclosure, in the step S2, the homogenizing is carried out at a rotating speed of 1,000 rpm to 3,000 rpm, and lasts for 0.5 hour to 3 hours.

In some embodiments of the present disclosure, the step S2 further comprises performing the homogenizing when preparing the acidic mixed solution of the Alpiniae Officinarum Rhizome precipitate; and preferably, the homogenizing is carried out at a rotating speed of 1,000 rpm to 3,000 rpm, and lasts for 0.5 hour to 3 hours.

In some embodiments of the present disclosure, a mass ratio of the emulsifier, the filler and the Alpiniae Officinarum Rhizome is (3 to 12):(17 to 48):(40 to 80); and preferably (5 to 10):(20 to 35):(45 to 70).

In some embodiments of the present disclosure, the emulsifier comprises at least one of sodium stearoyl lactate, calcium stearoyl lactate, monoglycerol fatty acid ester, polyglycerol ester, sucrose fatty acid ester, succinylated monoglycerides, propylene glycol alginate, soybean lecithin and propylene glycol fatty acid ester.

In some embodiments of the present disclosure, the filler comprises at least one of starch, maltodextrin, β-cyclodextrin, microcrystalline cellulose and erythritol.

In some embodiments of the present disclosure, in the step S3, the grinding is carried out at a rotating speed of 2,800 rpm to 3,200 rpm, and lasts for 1 hour to 5 hours (such as 2 hours, 3 hours, and 4 hours, and the like); and preferably, the grinding is carried out at 35° C. to 40° C. (such as 36° C., 37° C., 38° C. and 39° C.) in cooperation with zirconia beads with a diameter of 0.3 mm to 0.4 mm.

In some embodiments of the present disclosure, in the step S3, the drying is spray drying; and preferably, the drying is carried out at a temperature of 165° C. to 185° C.

In a second aspect of the present disclosure, an Alpiniae Officinarum Rhizome extract prepared by the preparation method of the Alpiniae Officinarum Rhizome extract is provided.

In some embodiments of the present disclosure, raw materials for preparing the Alpiniae Officinarum Rhizome extract comprise: the Alpiniae Officinarum Rhizome, the emulsifier and the filler with a mass ratio of (40 to 80):(3 to 12):(17 to 48); and preferably (45 to 70):(5 to 10):(20 to 35).

In some embodiments of the present disclosure, the Alpiniae Officinarum Rhizome extract is granular; preferably, an average particle diameter D₅₀ of the Alpiniae Officinarum Rhizome extract is 100 nm to 300 nm; such as 150 nm, 180 nm, 200 nm, 215 nm, 230 nm, 240 nm, and 250 nm, and the like.

In some embodiments of the present disclosure, a content of galangin in the Alpiniae Officinarum Rhizome extract is 0.7 wt % to 1.0 wt % of the Alpiniae Officinarum Rhizome superfine powder.

In a third aspect of the present disclosure, a composition is provided, which comprises the Alpiniae Officinarum Rhizome extract.

In some embodiments of the present disclosure, the composition comprises a traditional Chinese medicine composition for improving gastrointestinal inflammation.

In some embodiments of the present disclosure, the composition comprises a health care product and a food for assisting in protecting gastric mucosal injury.

The present disclosure has the below beneficial effects.

The in-situ emulsification preparation technology of the Alpiniae Officinarum Rhizome extract of the present disclosure solves the problems of a large amount of solvent and harsh processing condition of a traditional technology, and simultaneously reduces the problem of strong acrid taste of the Alpiniae Officinarum Rhizome extract, thus being suitable for industrial popularization and application.

The Alpiniae Officinarum Rhizome extract prepared by the present disclosure has a low acrid taste, a high content of galangin and high stability.

### DESCRIPTION OF THE DRAWINGS

FIG. 1 is a particle diameter distribution diagram of Alpiniae Officinarum Rhizome superfine powder of the present disclosure.

FIG. 2 is a particle diameter distribution diagram of the Alpiniae Officinarum Rhizome extract prepared in Example 1 of the present disclosure.

FIG. 3 is a particle diameter distribution diagram of the Alpiniae Officinarum Rhizome extract prepared in Example 2 of the present disclosure.

FIG. 4 is a particle diameter distribution diagram of the Alpiniae Officinarum Rhizome extract prepared in Example 3 of the present disclosure.

FIG. 5 is a particle diameter distribution diagram of the Alpiniae Officinarum Rhizome extract prepared in Example 4 of the present disclosure.

### DETAILED DESCRIPTION

The contents of the present disclosure are further described in detail hereinafter with reference to specific examples. Unless otherwise specified, raw materials, reagents or devices used in examples and comparative examples may all be commercially available conventionally, or obtained through the prior art. Unless otherwise specified, experiment or test methods are all conventional methods in the art.

### Example 1

In this example, an Alpiniae Officinarum Rhizome extract was prepared, and raw materials for preparing the Alpiniae Officinarum Rhizome extract comprised the following ingredients in percentage by weight: 80% of Alpiniae Officinarum Rhizome, 3% of emulsifier (monoglycerol fatty acid ester and polyglycerol ester, with a mass ratio of 1:1) and 17% of maltodextrin. A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with a citric acid aqueous solution with a pH value of 3.5 to 5.5 according to a solid-liquid ratio (g/mL) of 1:8, and sealed and stood for 30 minutes to obtain an acidic mixed solution of Alpiniae Officinarum Rhizome superfine powder.

(3) The acidic mixed solution of Alpiniae Officinarum Rhizome superfine powder was added with peanut oil according to a solid-liquid ratio (g/mL) of Alpiniae Officinarum Rhizome superfine powder and peanut oil of 1:1, stirred evenly, sealed and stood for 3 hours, and centrifuged to remove the peanut oil to obtain an Alpiniae Officinarum Rhizome precipitate.

(4) The Alpiniae Officinarum Rhizome precipitate was added with a citric acid aqueous solution with a pH value of 4.0 according to a solid-liquid ratio (g/mL) of 1:8, homogenized at 3,000 r/min for 0.5 hour, added with an emulsifier and a filler according to above ingredient amounts, and continuously homogenized at 3,000 r/min for 0.5 hour to obtain a homogeneous solution of Alpiniae Officinarum Rhizome.

(5) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(6) The grinding solution of Alpiniae Officinarum Rhizome was subjected to spray drying at 165° C. to obtain the Alpiniae Officinarum Rhizome extract.

### Example 2

In this example, an Alpiniae Officinarum Rhizome extract was prepared, and raw materials for preparing the Alpiniae Officinarum Rhizome extract comprised the following ingredients in percentage by weight: 40% of Alpiniae Officinarum Rhizome, 12% of monoglycerol fatty acid ester and 48% of microcrystalline cellulose. A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with a citric acid aqueous solution with a pH value of 3.5 to 5.5 according to a solid-liquid ratio (g/mL) of 1:6, and sealed and stood for 30 minutes to obtain an acidic mixed solution of Alpiniae Officinarum Rhizome superfine powder.

(3) The acidic mixed solution of Alpiniae Officinarum Rhizome superfine powder was added with peanut oil according to a solid-liquid ratio (g/mL) of Alpiniae Officinarum Rhizome superfine powder and peanut oil of 1:0.5, stirred evenly, sealed and stood for 3 hours, and centrifuged to remove the peanut oil to obtain an Alpiniae Officinarum Rhizome precipitate.

(4) The Alpiniae Officinarum Rhizome precipitate was added with a citric acid aqueous solution with a pH value of 4.0 according to a solid-liquid ratio (g/mL) of 1:8, homogenized at 3,000 r/min for 0.5 hour, added with an emulsifier and a filler according to above ingredient amounts, and continuously homogenized at 3,000 r/min for 0.5 hour to obtain a homogeneous solution of Alpiniae Officinarum Rhizome.

(5) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(6) The grinding solution of Alpiniae Officinarum Rhizome was subjected to spray drying at 175° C. to obtain the Alpiniae Officinarum Rhizome extract.

### Example 3

In this example, an Alpiniae Officinarum Rhizome extract was prepared, and raw materials for preparing the Alpiniae Officinarum Rhizome extract comprised the following ingredients in percentage by weight: 60% of Alpiniae Officinarum Rhizome, 5% of sucrose fatty acid ester and 35% of filler (starch and erythritol, with a mass ratio 1:1). A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with a citric acid aqueous solution with a pH value of 3.5 to 5.5 according to a solid-liquid ratio (g/mL) of 1:8, and sealed and stood for 30 minutes to obtain an acidic mixed solution of Alpiniae Officinarum Rhizome superfine powder.

(3) The acidic mixed solution of Alpiniae Officinarum Rhizome superfine powder was added with soybean oil according to a solid-liquid ratio (g/mL) of Alpiniae Officinarum Rhizome superfine powder and soybean oil of 1:3, stirred evenly, sealed and stood for 3 hours, and centrifuged to remove the soybean oil to obtain an Alpiniae Officinarum Rhizome precipitate.

(4) The Alpiniae Officinarum Rhizome precipitate was added with a citric acid aqueous solution with a pH value of 4.0 according to a solid-liquid ratio (g/mL) of 1:12, homogenized at 3,000 r/min for 0.5 hour, added with an emulsifier and a filler according to above ingredient amounts, and continuously homogenized at 3,000 r/min for 0.5 hour to obtain a homogeneous solution of Alpiniae Officinarum Rhizome.

(5) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(6) The grinding solution of Alpiniae Officinarum Rhizome was subjected to spray drying at 170° C. to obtain the Alpiniae Officinarum Rhizome extract.

### Example 4

In this example, an Alpiniae Officinarum Rhizome extract was prepared, and raw materials for preparing the Alpiniae Officinarum Rhizome extract comprised the following ingredients in percentage by weight: 60% of Alpiniae Officinarum Rhizome, 3% of propylene glycol fatty acid ester and 37% of β-cyclodextrin. A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with a tartaric acid aqueous solution with a pH value of 3.5 to 5.5 according to a solid-liquid ratio (g/mL) of 1:8, and sealed and stood for 30 minutes to obtain an acidic mixed solution of Alpiniae Officinarum Rhizome superfine powder.

(3) The acidic mixed solution of Alpiniae Officinarum Rhizome superfine powder was added with safflower seed oil according to a solid-liquid ratio (g/mL) of Alpiniae Officinarum Rhizome superfine powder and peanut oil of 1:3, stirred evenly, sealed and stood for 3 hours, and centrifuged to remove the safflower seed to obtain an Alpiniae Officinarum Rhizome precipitate.

(4) The Alpiniae Officinarum Rhizome precipitate was added with a tartaric acid aqueous solution with a pH value of 4.0 according to a solid-liquid ratio (g/mL) of 1:6, homogenized at 3,000 r/min for 0.5 hour, added with an emulsifier and a filler according to above ingredient amounts, and continuously homogenized at 3,000 r/min for 0.5 hour to obtain a homogeneous solution of Alpiniae Officinarum Rhizome.

(5) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(6) The grinding solution of Alpiniae Officinarum Rhizome was subjected to spray drying at 165° C. to obtain the Alpiniae Officinarum Rhizome extract.

### Comparative Example 1

In this comparative example, an Alpiniae Officinarum Rhizome extract was prepared, and raw materials of Alpiniae Officinarum Rhizome comprised the following ingredients in percentage by weight: 80% of Alpiniae Officinarum Rhizome, 3% of emulsifier (monoglycerol fatty acid ester and polyglycerol ester, with a mass ratio of 1:1) and 17% of maltodextrin. A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with ethyl acetate according to a solid-liquid ratio (g/mL) of 1:1, stirred evenly, sealed and stood for 3 hours, and centrifuged to remove the ethyl acetate to obtain an Alpiniae Officinarum Rhizome precipitate.

(3) The Alpiniae Officinarum Rhizome precipitate was added with a citric acid aqueous solution with a pH value of 4.0 according to a solid-liquid ratio (g/mL) of 1:8, homogenized at 3,000 r/min for 0.5 hour, added with an emulsifier and a filler according to above ingredient amounts, and continuously homogenized at 3,000 r/min for 0.5 hour to obtain a homogeneous solution of Alpiniae Officinarum Rhizome.

(4) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(5) The grinding solution of Alpiniae Officinarum Rhizome was subjected to spray drying at 165° C. to obtain the Alpiniae Officinarum Rhizome extract.

### Comparative Example 2

In this comparative example, an Alpiniae Officinarum Rhizome extract was prepared, and raw materials of Alpiniae Officinarum Rhizome comprised the following ingredients in percentage by weight: 80% of Alpiniae Officinarum Rhizome, 3% of emulsifier (monoglycerol fatty acid ester and polyglycerol ester, with a mass ratio of 1:1) and 17% of maltodextrin. A specific process was as follows.

(1) A medicinal material of Alpiniae Officinarum Rhizome was crushed, sieved with an 80-mesh sieve, and then subjected to superfine crushing to obtain Alpiniae Officinarum Rhizome superfine powder.

(2) The Alpiniae Officinarum Rhizome superfine powder was added with n-hexane according to a solid-liquid ratio (g/mL) of 1:1, stirred evenly, sealed and stood for 3 hours, and centrifuged to remove the n-hexane to obtain an Alpiniae Officinarum Rhizome precipitate.

(3) The Alpiniae Officinarum Rhizome precipitate was added with a citric acid aqueous solution with a pH value of 4.0 according to a solid-liquid ratio (g/mL) of 1:8, homogenized at 3,000 r/min for 0.5 hour, added with an emulsifier and a filler according to the above ingredient amounts, and continuously homogenized at 3,000 r/min for 0.5 hour to obtain a homogeneous solution of Alpiniae Officinarum Rhizome.

(4) The homogeneous solution of Alpiniae Officinarum Rhizome was ground with zirconia beads of 0.3 mm to 0.4 mm at a rotating speed of 3,000 r/min and a temperature of 37° C. for 3 hours to obtain a grinding solution of Alpiniae Officinarum Rhizome.

(5) The grinding solution of Alpiniae Officinarum Rhizome was subjected to spray drying at 165° C. to obtain the Alpiniae Officinarum Rhizome extract.

### Comparative Example 3

In this comparative example, an Alpiniae Officinarum Rhizome extract was prepared, which differed from Example 1 in that the method in Comparative Example 3 did not comprise step (2), and other steps were the same as those in Example L.

### Experimental Example

1. In this experimental example, a particle diameter of an Alpiniae Officinarum Rhizome extract was tested. A specific process was as follows.

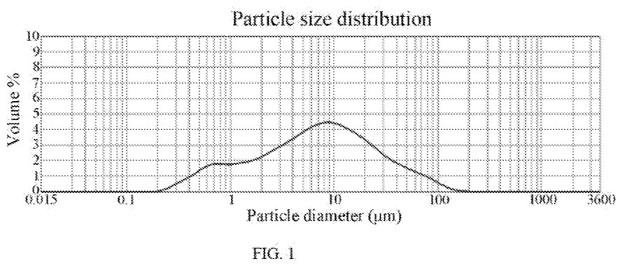

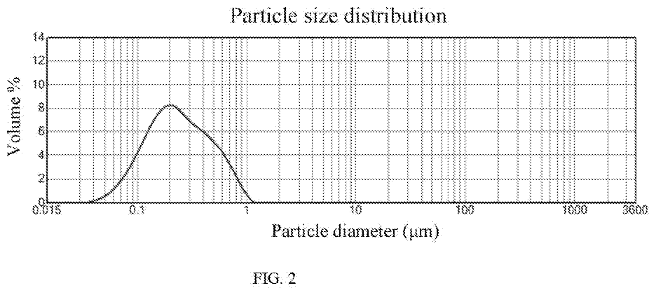

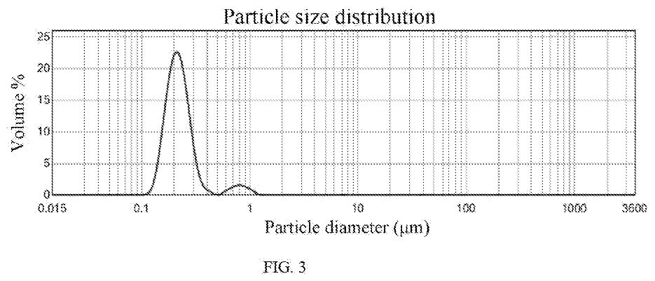

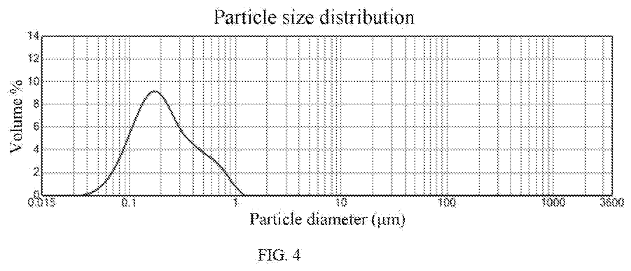

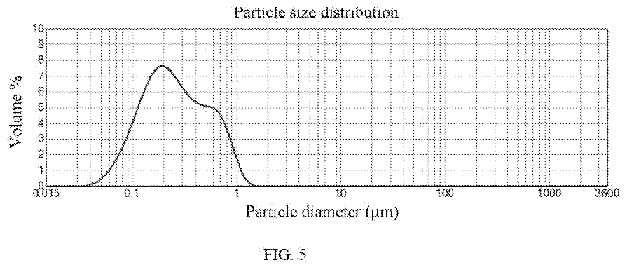

Particle diameters of the Alpiniae Officinarum Rhizome extracts prepared in the above examples were tested with a laser particle size analyzer (LT3600Plus, Linkoptik Instrument Co., Ltd, ZHUHAI, China) by a method comprising adding 100 mL of purified water into 0.1 g of Alpiniae Officinarum Rhizome extract powder into for dilution, homogenizing and stirring a resulting mixture evenly, and then testing their particle diameters directly on the machine. Results were shown in Table 1 and FIG. 1 to FIG. 5.

TABLE 1

| Sample | Average particle diameter D₅₀/(nm) |
|---|---|
| Alpiniae Officinarum | 7011 |
| Rhizome superfine | |
| powder | |
| Example 1 | 229 |
| Example 2 | 217 |
| Example 3 | 198 |
| Example 4 | 244 |

It can be seen that the Alpiniae Officinarum Rhizome superfine powder is micron-sized, a particle diameter of a powder body is relatively large. However, after the treatment by the technology of the present disclosure, a particle diameter of the powder body of the raw materials of Alpiniae Officinarum Rhizome is obviously reduced, which is beneficial for an application of the raw material in brewing liquid preparations such as a solid drink and a liquid drink.

2. In this experimental example, an acrid taste of the Alpiniae Officinarum Rhizome extract was also tested. A specific process was as follows.

A sensory on method was used for testing, 2 g of Alpiniae Officinarum Rhizome extracts prepared in Examples 1 to 4 and Comparative Examples 1 to 2, 1 g of Alpiniae Officinarum Rhizome superfine powder and 0.1 g of commercially available Alpiniae Officinarum Rhizome ethanol extract were respectively added with 200 mL of hot water (70° C.), stirred evenly, and placed for 20 minutes before oral administration, 10 subjects were selected for sensory testing, 10 mL of the solution were taken orally each time, and an acrid taste score of the solution was recorded according to a scoring rule that 1 score was recorded in the case of no acrid taste, 2 scores were recorded in the case of slight acrid taste, 3 scores were recorded in the case of obvious acrid taste, and 4 scores were recorded in the case of strong acrid taste. Scoring results were shown in Table 2.

TABLE 2

| | Commercially | Alpiniae | | | | | | |
|---|---|---|---|---|---|---|---|---|
| | available | Officinarum | | | | | | |
| | Alpiniae | Rhizome | | | | | | |
| Serial | Officinarum | superfine | Example | Example | Example | Example | Comparative | Comparative |
| number | Rhizome extract | powder | 1 | 2 | 3 | 4 | Example 1 | Example 2 |
| 1 | 4 | 4 | 2 | 2 | 1 | 1 | 2 | 3 |
| 2 | 4 | 3 | 1 | 2 | 1 | 1 | 2 | 3 |
| 3 | 4 | 4 | 2 | 3 | 2 | 2 | 3 | 3 |
| 4 | 4 | 3 | 2 | 2 | 1 | 1 | 2 | 2 |
| 5 | 4 | 4 | 2 | 2 | 1 | 2 | 3 | 3 |
| 6 | 3 | 3 | 1 | 2 | 1 | 1 | 2 | 2 |
| 7 | 4 | 4 | 2 | 2 | 2 | 2 | 2 | 2 |
| 8 | 3 | 3 | 1 | 2 | 1 | 1 | 3 | 3 |
| 9 | 4 | 4 | 2 | 2 | 1 | 1 | 2 | 2 |
| 10 | 4 | 3 | 1 | 2 | 1 | 1 | 2 | 3 |
| Average | 3.8 | 3.5 | 1.6 | 2.1 | 1.2 | 1.3 | 2.3 | 2.6 |
| value | | | | | | | | |

It can be seen that, compared with the commercially available Alpiniae Officinarum Rhizome extract and Alpiniae Officinarum Rhizome superfine powder, the acrid taste of the Alpiniae Officinarum Rhizome extracts in Examples 1 to 4 are obviously reduced, indicating that the Alpiniae Officinarum Rhizome extracts prepared in the examples of the present disclosure have good advantages.

3. In this experimental example, a content of galangin in the Alpiniae Officinarum Rhizome extract was tested. A specific process was as follows.

Preparation of test solution: about 0.2 g of dried powder (sieved by an 80-mesh sieve) of the Alpiniae Officinarum Rhizome extract to be tested was accurately weighed, placed in a round-bottomed flask, accurately added with 50 mL of methanol, weighed, heated and refluxed for 1 hour, cooled by standing, and then weighed again, a lost weight was supplemented with methanol, the obtained mixture was shaken evenly and filtered, and the subsequent filtrate was taken to obtain the test solution.

Chromatographic conditions: a stationary phase was octadecylsilane bonded silica gel, at 40° C.; a mobile phase was methanol: 0.2% phosphoric acid aqueous solution=55:45 (0 minute to 45 minutes, equidegree): a detection wavelength was 266 nm, and 10 ng/mL, 20 μg/mL, 40 μg/mL, 60 μg/ml and 80 μg/mL galangin standard solutions were used to establish standard curves, and the contents of galangin were detected.

TABLE 3

| Serial number | Content of galangin (%) |
|---|---|
| Alpiniae Officinarum | 0.71 |
| Rhizome superfine | |
| powder | |
| Example 1 | 0.79 |
| Example 2 | 0.75 |
| Example 3 | 0.86 |
| Example 4 | 0.82 |
| Comparative Example 1 | 0.24 |
| Comparative Example 2 | 0.27 |
| Comparative Example 3 | 0.185 |

The above examples are the preferred implementations of the present disclosure, but the implementations of the present disclosure are not limited by the above examples. Any other changes, modifications, substitutions, combinations, and simplifications made without departing from the spirit and principle of the present disclosure should be equivalent substitute modes, and should be included in the scope of protection of the present disclosure.

1. A preparation method of an Alpiniae Officinarum Rhizome extract, comprising the following steps of:
S1: adding vegetable oil into an acidic mixed solution of Alpiniae Officinarum Rhizome superfine powder and stirring a resulting mixture, and then removing the vegetable oil to obtain an Alpiniae Officinarum Rhizome precipitate;
S2: adding an emulsifier and a filler into an acidic mixed solution of the Alpiniae Officinarum Rhizome precipitate, and homogenizing a resulting mixture to obtain a homogeneous solution of Alpiniae Officinarum Rhizome; and
S3: grinding and then drying the homogeneous solution of Alpiniae Officinarum Rhizome to obtain the Alpiniae Officinarum Rhizome extract.

2. The preparation method of the Alpiniae Officinarum according to claim 1, wherein, in the step S1, a preparation method of the acidic mixed solution of the Alpiniae Officinarum Rhizome superfine powder comprises the following steps of: crushing the Alpiniae Officinarum Rhizome, sieving a crushed Alpiniae Officinarum Rhizome with an 80-mesh to 150-mesh sieve, and then carrying out superfine crushing on a sieved Alpiniae Officinarum Rhizome to prepare the Alpiniae Officinarum Rhizome superfine powder; and adding an acidic solution into the Alpiniae Officinarum Rhizome superfine powder according to a solid-liquid ratio of 1 g: (6 to 12) mL, and sealing and standing a resulting mixture to prepare the acidic mixed solution of the Alpiniae Officinarum Rhizome superfine powder.

3. The preparation method of the Alpiniae Officinarum Rhizome extract according to claim 2, wherein a pH value of the acidic solution is 3.5 to 5.5.

4. The preparation method of the Alpiniae Officinarum Rhizome extract according to claim 3, wherein the pH value of the acidic solution is regulated to be 3.5 to 5.5 with an acidity regulator.

5. The preparation method of the Alpiniae Officinarum Rhizome extract according to claim 4, wherein the acidity regulator comprises at least one of citric acid, tartaric acid, malic acid and acetic acid.

6. The preparation method of the Alpiniae Officinarum Rhizome extract according to claim 1, wherein, in the step S1, a solid-liquid ratio of the Alpiniae Officinarum Rhizome superfine powder to the vegetable oil is 1:(0.5 to 3).

7. The preparation method of the Alpiniae Officinarum Rhizome extract according to claim 1, wherein the vegetable oil comprises at least one of peanut oil, olive oil, safflower seed oil, soybean oil, corn oil and rapeseed oil.

8. The preparation method of the Alpiniae Officinarum Rhizome extract according to claim 1, wherein in the step S2, a preparation method of the acidic mixed solution of the Alpiniae Officinarum Rhizome precipitate comprises the following step of: adding the Alpiniae Officinarum Rhizome precipitate into an acidic solution according to a solid-liquid ratio of 1 g: (6 to 12) mL.

9. The preparation method of the Alpiniae Officinarum Rhizome extract according to claim 8, wherein a pH value of the acidic solution is 3.5 to 5.5.

10. The preparation method of the Alpiniae Officinarum Rhizome extract according to claim 9, wherein the pH value of the acidic solution is regulated to be 3.5 to 5.5 with an acidity regulator.

11. The preparation method of the Alpiniae Officinarum Rhizome extract according to claim 10, wherein the acidity regulator comprises at least one of citric acid, tartaric acid, malic acid and acetic acid.

12. The preparation method of the Alpiniae Officinarum Rhizome extract according to claim 1, wherein a mass ratio of the emulsifier, the filler and the Alpiniae Officinarum Rhizome is (3 to 12):(17 to 48):(40 to 80).

13. The preparation method of the Alpiniae Officinarum Rhizome extract according to claim 12, wherein the emulsifier comprises at least one of sodium stearoyl lactate, calcium stearoyl lactate, monoglycerol fatty acid ester, polyglycerol ester, sucrose fatty acid ester, succinylated monoglycerides, propylene glycol alginate, soybean lecithin and propylene glycol fatty acid ester.

14. The preparation method of the Alpiniae Officinarum Rhizome extract according to claim 12, wherein the filler comprises at least one of starch, maltodextrin, p-cyclodextrin, microcrystalline cellulose and erythritol.

15. The preparation method of the Alpiniae Officinarum Rhizome extract according to claim 1, wherein, in the step S2, a solid-liquid ratio of the acidic mixed solution of the Alpiniae Officinarum Rhizome precipitate is 1 g: (6 to 12) mL.

16. The preparation method of the Alpiniae Officinarum Rhizome extract according to claim 1, in the step S2, the homogenizing is carried out at a rotating speed of 1,000 rpm to 3,000 rpm, and lasts for 0.5 hour to 3 hours.

17. An Alpiniae Officinarum Rhizome extract prepared by the preparation method of the Alpiniae Officinarum Rhizome extract according to claim 1.

18. The Alpiniae Officinarum Rhizome extract according to claim 17, wherein an average particle diameter D₅₀ of the Alpiniae Officinarum Rhizome extract is 100 nm to 300 nm.

19. The Alpiniae Officinarum Rhizome extract according to claim 17, wherein a content of galangin in the Alpiniae Officinarum Rhizome extract is 0.7 we % to 1.0 wt % of the Alpiniae Officinarum Rhizome superfine powder.

20. A composition, comprising the Alpiniae Officinarum Rhizome extract according to claim 17.
